(19) United States
(12) Patent Application Publication
(10) Pub. No.: US 2008/0160070 A1
(43) Pub. Date: Jul. 3, 2008

(54) TRANSDERMAL VITAMIN B12 DELIVERY PATCH

(75) Inventor: David Crawford, Wadsworth, OH (US)

Correspondence Address: DAVID P DURESKA;BUCKINGHAM DOOLITTLE & BURROUGHS, LLP, 4518 FULTON DRIVE, NW, P O BOX 35548, CANTON, OH, 44735-5548, US

(73) Assignee: Nexagen USA LLC, Akron, OH (US)

(21) Appl. No.: 11/961,234

(22) Filed: Dec. 20, 2007

Related U.S. Application Data
(60) Provisional application No. 60/877,330, filed on Dec. 27, 2006.

(51) Int. Cl. A61K 9/70; A61K 31/714

(52) U.S. Cl. 424/449; 514/52

(57) ABSTRACT

A transdermal vitamin B12 delivery patch is applied to the skin of a user for the delivery of vitamin B12 to the bloodstream of the user. The patch includes a fabric backing and a skin-adhesive polymer matrix that is attached to one side of the fabric backing. The matrix contains a vitamin B12 compound. The vitamin B12 compound diffuses from the matrix through the stratum corneum layer of the user's skin, through the dermis layer of the skin, and into the user's bloodstream.

### CROSS-REFERENCE TO RELATED APPLICATION

This application claims the benefit of U.S. Provisional Patent Application Ser. No. 60/877,330, which was filed on Dec. 27, 2006.

### BACKGROUND OF THE INVENTION

1. Technical Field

The present invention generally relates to transdermal delivery of vitamin B12 through a patch made of a base fabric, a skin-adhesive polymer matrix on one side of the base fabric, and a B12 compound.

2. Background Art

Vitamin B12 is an essential vitamin, working in blood and nerve cells. Vitamin B12 is commonly called cobalamin, as it contains cobalt. Vitamin B12 is found naturally in dietary sources such as meat, dairy products and eggs. Recommendations for daily intakes of vitamin B12 are established by the Institute of Medicine of the National Academy of Sciences. Certain individuals need an increased amount of vitamin B12, such as breastfeeding women, who may need additional amounts of vitamin B12 to ensure an adequate supply of breast milk.

An individual's inability to absorb adequate quantities of vitamin B12 from his/her diet may lead to hematological and neurological complications. In some individuals, particularly seniors, vitamin B12 deficiencies are usually due to a condition called pernicious anemia. In pernicious anemia, the stomach does not make intrinsic factor, which is a substance necessary for absorbing vitamin B12 into the body. In addition to seniors, this condition is somewhat common among persons of Scandinavia, Irish or English backgrounds. Less often, vitamin B12 deficiency is found in strict vegetarians and those who had their stomach or ileum surgically removed. Vitamin B12 uptake may also be impaired among people using antacids and reflux inhibitors.

While many people who suffer from vitamin B12 deficiency go undiagnosed, more and more health practitioners and consumers are becoming aware of the dramatic benefit vitamin B12 provides not only in treating pernicious anemia, but also in relieving fatigue, cognitive decline, and depression. Vitamin B12 has a wide dosage window, with an extremely low tendency for allergic reaction. It is nontoxic at high concentrations, and generally is considered safe to use. Supplementation of vitamin B12 is currently accomplished, in decreasing order of absorption efficiency, through: intramuscular injection; intra nasal gel; and oral capsules or sublingual tablets.

Initial therapy for a substantial vitamin B12 deficiency generally requires intramuscular injections, because the human body is very inefficient at absorbing vitamin B12 dietary supplements. Less intrusive vitamin B12 delivery means may replace the injections once the vitamin B12 deficiency has been corrected through a schedule of vitamin B12 injections. Currently, nasal gels are popular, often being preferred over injections.

When an individual suffers from vitamin B12 deficiency, it is recommended that he or she maintains healthy levels of vitamin B12 through supplemental vitamin therapy, which typically is administered weekly in the nasal gel form or monthly as intramuscular injections. Although vitamin B12 is widely available in oral form, many patients who have conditions such as multiple sclerosis or inflammatory bowel disease may have severe deficiencies of vitamin B12 due to their compromised ability to absorb it through the gastrointestinal system. Although intramuscular injections and nasal gel delivery of vitamin B12 provide a means for delivering vitamin B12 in a manner that typically is better absorbed, a less invasive means for delivery of vitamin B12 is desired. The present invention provides such a means.

### SUMMARY OF THE INVENTION

One objective of the present invention is to provide a transdermal patch for the delivery of the vitamin B12 to a user.

Another objective of the present invention is to provide a transdermal patch for delivery of the vitamin B12 without the trauma or invasion experienced with prior art intramuscular injections and nasal gels, while providing more efficient absorption of the vitamin than the absorption through the gastrointestinal system that is provided by the prior art.

Yet another objective of the present invention is to provide a patch for the delivery of the vitamin B12 to a user with an ease of use by the end user that is increased over prior art delivery methods of vitamin B12.

These objectives and others are obtained by the transdermal vitamin B12 delivery patch of the present invention. The transdermal vitamin B12 delivery patch is applied to the skin of a user for the delivery of vitamin B12 to the bloodstream of the user. The patch includes a fabric backing and a skin-adhesive polymer matrix that is attached to one side of the fabric backing. The matrix contains a vitamin B12 compound. The vitamin B12 compound diffuses from the matrix through the stratum corneum layer of the user's skin, through the dermis layer of the skin, and into the user's bloodstream.

In preferred embodiments, permeation enhancers are employed to help improve the diffusion of the vitamin B12 compound from within the skin-adhesive polymer matrix through the stratum corneum and the dermis. In a particularly preferred embodiment, a chemical stabilizer is also employed to retard or prevent ultraviolet light-induced degradation of the vitamin B12 compound.

These and other objectives and advantages of the present invention will become more readily apparent from a reading of the following detailed description and the accompanying drawings, and from the invention as set forth in the appended claims.

### BRIEF DESCRIPTION OF THE DRAWINGS

The preferred embodiments of the present invention, illustrative of the best modes in which applicant has contemplated applying the principles of the invention, are set forth in the following description, are shown in the drawings, and are particularly and distinctly pointed out and set forth in the appended claims.

FIG. 1 is a top plan view of an exemplary embodiment of a vitamin B12 transdermal delivery patch of the present invention;

FIG. 2 is a cross-sectional view of the patch shown in FIG. 1, taken along the line 2-2 in FIG. 1, with the thickness of the patch shown in FIG. 1 being generally enlarged;

FIG. 3 is a graph of vitamin B12 concentration in the bloodstream versus time, pursuant to vitamin B12 delivery by an exemplary embodiment of the vitamin B12 transdermal delivery patch of the present invention, averaged for 6 subject trials; and

FIG. 4 is a representation of the graph of FIG. 3 shown with standard deviations.

Similar numerals refer to similar parts throughout the drawings.

### DETAILED DESCRIPTION OF THE INVENTION

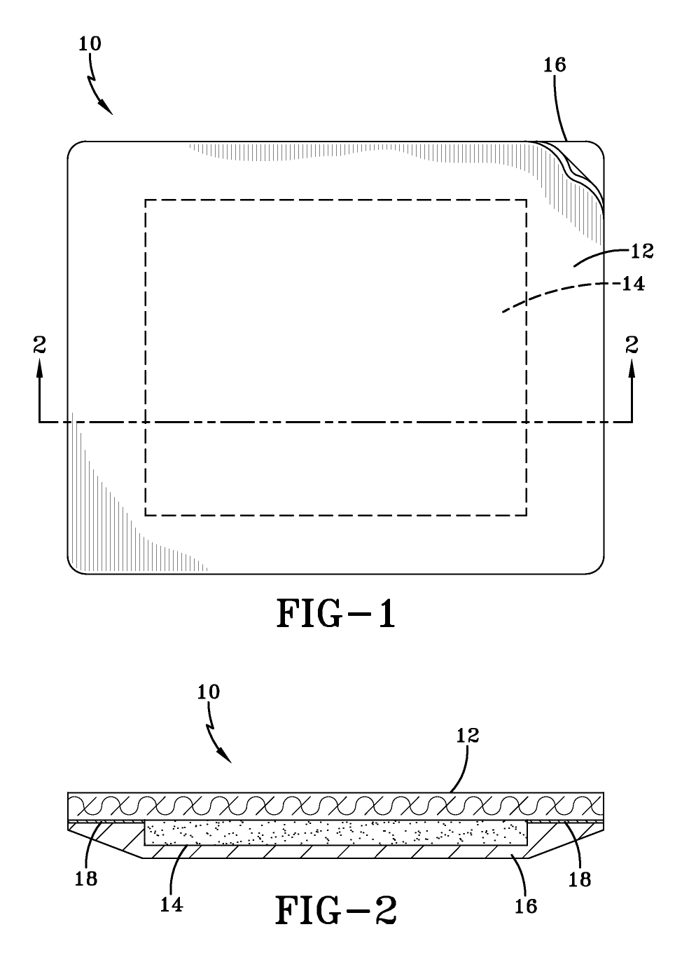

Referring now to the drawings, wherein the showings are for purposes of illustrating preferred embodiments of the invention and not for purposes of limiting the same, FIGS. 1 and 2 illustrate exemplary embodiments of the present invention for a transdermal patch for the delivery of vitamin B12, which is indicated generally at 10.

Patch 10 includes a fabric backing 12, which carries a skin-adhesive polymer matrix 14 that is attached to the fabric backing. Skin-adhesive polymer matrix 14 contains a vitamin B12 compound, and when the skin-adhesive polymer matrix is adhered to the skin, the vitamin B12 compound diffuses from within the skin-adhesive polymer matrix through the stratum corneum layer of the epidermis, though the dermis into the microvascular and thus enters the bloodstream of the user. The vitamin B12 compound may be distributed uniformly throughout skin-adhesive polymer matrix 14, or may have varying concentrations throughout the matrix. For example, in one exemplary embodiment of patch 10, higher concentrations of vitamin B12 compound may exist adjacent or near the area of adhesive matrix 14 that is in direct contact with the user's skin, and lower concentrations of vitamin B12 compound may exist near the area that is adjacent or near the fabric backing. In another exemplary embodiment of the present invention, higher concentrations of vitamin B12 compound exist near the center of patch 10, and lower concentrations of the compound exist near the edges of the patch. In yet another exemplary embodiment, vitamin B12 compound may be placed in a dotted pattern to provide high density areas of vitamin B12 compound along with optimized adhesive areas.

To protect skin-adhesive polymer matrix 14 and the vitamin B12 compound therein, the polymer matrix and compounds carried thereby preferably are sandwiched between fabric backing 12 and a suitable release liner 16. Release liner 16 serves to prevent polymer matrix 14 and vitamin B12 compound therein from inadvertently being displaced from fabric backing 12 before patch 10 is applied to the user's skin. Immediately prior to application to the skin, release liner 16 is removed from patch 10 so that polymer matrix 14, containing vitamin B12 compound, may be adhered directly to the user's skin, with fabric backing 12 serving to protect the polymer matrix from the external or opposite side of the patch. Preferably, release liner 16 is made of a material as known in the art, which enables the release liner to be removed from fabric backing 12 cleanly, with minimal displacement of skin-adhesive polymer matrix 14 and the vitamin B12 compound therein. Release liner 16 may be removed and patch 10 thereby adhered to the skin by the user themselves, due to the relative ease of use of the patch of the present invention, or a third party may apply the patch to the user.

Fabric backing 12 may be sized similarly to that of polymer matrix 14, or the fabric backing may be sized larger than the polymer matrix. Fabric backing 12 may include a suitable adhesive layer or area 18 which releasably adheres release liner 16 to the fabric backing when the fabric backing and the release liner are sized larger than polymer matrix 14. Adhesive layer 18 may also assist in enabling patch 10 to adhere to the user's skin when release liner 16 is removed, and optionally may extend across fabric backing 12 between the fabric backing and polymer matrix 14 to help secure the polymer matrix to the fabric backing.

After application of patch 10 to the user's skin, essentially no limitations exist as to the length of time that the patch can remain in contact with the user's skin. Since the amount of vitamin B12 compound in polymer matrix 14 will decrease as it is absorbed into the user's skin, patch 10 ideally is removed from the user's skin before the amount of vitamin B12 compound existing in the polymer matrix decreases to an amount that is no longer effective to the user. It is to be understood that the amount of vitamin B12 compound initially carried in polymer matrix 14 will affect the length of time patch 10 will be effective once the patch is applied to the user's skin. For example, in an exemplary embodiment of the invention, polymer matrix 14 contains a vitamin B12 compound that in turn contains 1,500 micrograms of cyanocobalamin, which preferably is used as a vitamin supplement. In such an embodiment, patch 10 should be removed after approximately 3 days, and after that time replaced with a new patch for continued absorption of vitamin B12 compound into the user's skin. However, patch 10 may optionally be left on longer than, or removed sooner than, the length of time that is necessary or recommended for complete diffusion of the vitamin B12 compound into the user's skin.

Fabric backing 12 may be made from any suitable material, which preferably is selected to be durable, comfortable and clean. For example, woven, non-woven, scrim, ribbon, composite or sheet fabric may be employed for fabric backing 12. Preferred materials for fabric backing 12 include polyester, polyethylene, vinyl, and combinations thereof. A particularly preferred material for fabric backing 12 is a foam fabric comprised of a fine-celled, irradiation cross-linked polyolefin. In certain alternative embodiments of the invention, it may be required that patch 10, and thus fabric backing 12, be sterile before application to a user's skin.

Fabric backing 12 may be of any color, size, shape, configuration, pattern, or texture. In a preferred embodiment, fabric backing 12 is made of a material that is translucent, so that the user's skin tone shows through patch 10. In another preferred embodiment, fabric backing 12 is a neutral color. Generally, the only limitation as to the physical size and thickness of patch 10 is that the patch must be of an appropriate size and thickness to carry the desired amount of vitamin B12 compound in skin-adhesive polymer matrix 14. It is generally preferred that patch 10 is of the minimum size necessary to effectively carry and diffuse the desired amount of vitamin B12 compound for particular users. For example, patch 10 may have a size of about four inches long by about three inches high by about one-eighth of an inch thick. Patch 10 may also be of any shape desired, including any number of common or unique shapes, such as square, circular, star-shaped, triangular, and so forth. A preferred shape of patch 10 is a rectangular shape.

Skin-adhesive polymer matrix 14 may be selected from any suitable polymer matrix able to carry and deliver the vitamin B12 compound through the stratum corneum layer of the epidermis, through the dermis into the microvascular, and thus into the bloodstream, and which is able to sufficiently adhere to the skin. Without limitation, polymer matrix 14 may be selected from polyisobutylene, polyoxyl hydrogenated castor oil, ethylene/vinyl acetate copolymer, methacrylate copolymer containing amminio groups, methacrylate copolymer containing amino groups, methacrylate polymer containing carboxyl groups, and mixtures of the foregoing. A particularly preferred polymer matrix 14 is an acrylic polymeric adhesive that includes between about 30% and 85% of a C₄-C₁₂ alkyl acrylate, between about 5% and 50% by weight of a C₁-C₄ alkyl acrylate hardening monomer, and between about 0.4% and 20% by weight of a functionalizing monomer that facilitates cross-linking. Preferably, the acrylic polymeric adhesive is chosen to cause the minimum irritation and agitation to the portion of the user's skin that is in contact with the adhesive.

As mentioned above, polymer matrix 14 serves to adhere patch 10 to the skin and deliver a vitamin B12 compound through the stratum corneum layer of the epidermis and through the dermis into the microvasculature. Preferably, skin-adhesive polymer matrix 14 carries from about 500 to about 2,000 micrograms of vitamin B12 compound, more preferably from about 1,000 to about 1,750 micrograms, and most preferably from about 1,400 to about 1,600 micrograms, with a most preferred embodiment containing about 1,500 micrograms. The vitamin B12 compound is preferably selected from biologically active forms, such as cyanocobalamin, aquacobalamin, hydroxocobalamin, nitritocobalamin, and combinations thereof. One preferred embodiment of the invention includes a vitamin B12 compound having the chemical name 5,6-dimethyl-benzimidazolyl cyanocobamide, which has a molecular formula of C63H88CoN14O14P, with a cobalt content of 4.34% and a molecular weight of 1355.39 u.

To improve the rate at which the vitamin B12 compound diffuses through the stratum corneum layer of the epidermis, permeation enhancers preferably are carried within skin-adhesive polymer matrix 14. The permeation enhancers may be selected from nicotinate compounds, fatty acids, fatty alcohols, terpenes, polyols, cyclic oligosaccharides, and combinations thereof. Preferably, the permeation enhancer is from about 0.001% to about 3% by weight of polymer matrix 14. Particularly preferred nicotinate compounds for the permeation enhancers include methyl nicotinate, benzyl nicotinate, 2-butoxyethyl nicotinate, isobutyl nicotinate, 1-carbamoylethyl nicotinate, phenyl nicotinate, and n-butyl nicotinate. Particularly preferred fatty acids for the permeation enhancers include oleic acid, undecanoic acid, valeric acid, heptanoic acid, pelargonic acid, capric acid, lauric acid, and eicosapentaenoic acid. Particularly preferred fatty alcohols for the permeation enhancers include octanol, nonanol, oleyl alcohol and decyl alcohol. Particularly preferred terpenes for the permeation enhancers include menthol, thymol, limonene, and terpineol. Particularly preferred polyols for the permeation enhancers include propylene glycol, polyethylene glycol, and glycerol. Particularly preferred cyclic oligosaccharides for the permeation enhancers include alpha cyclodextrin and (1-4)-linked glucopyranose.

The vitamin B12 compounds that are employed in accordance with this invention may degrade through exposure to ultraviolet (UV) light. Therefore, skin-adhesive polymer matrix 14 also preferably carries a UV stabilizer to retard and/or prevent ultraviolet light-induced degradation of the vitamin B12 compound. Particularly preferred UV stabilizers are selected from elemental selenium, sodium selenite, sodium selenate, sodium selenide, dibenzoyl diselenide, selenocystamine, selenobetaine, selenium-methylselenocysteine, selenomethionine, and combinations thereof. Preferably, the chemical stabilizer is from about 0.005 to about 2.0% of polymer matrix 14.

In accordance with the foregoing disclosure, a particularly preferred embodiment of patch 10 employs a skin adhesive polymer matrix 14 including 2-ethylhexyl acrylate, methyl acrylate and acrylamide. This matrix 14 carries the following preferred vitamin B12 compound, permeation enhancers, and chemical stabilizers:

| Ingredient | Name | Amount (by weight) |
|---|---|---|
| Vitamin B12 compound | Methylcobalamin | 0.25 to 1.5% |
| Permeation Enhancers | Methylnicotinate | 0.001 to 2.0% |
| | alphacyclodextrin | |
| Chemical Stabilizer | Sodium selenite | 0.005 to 0.25% |

Patches 10 according to this particularly preferred embodiment were employed in clinical experiments to analyze the rate at which the patch is able to deliver the vitamin B12 compound. Results of the experiment are provided below.

### Experimental

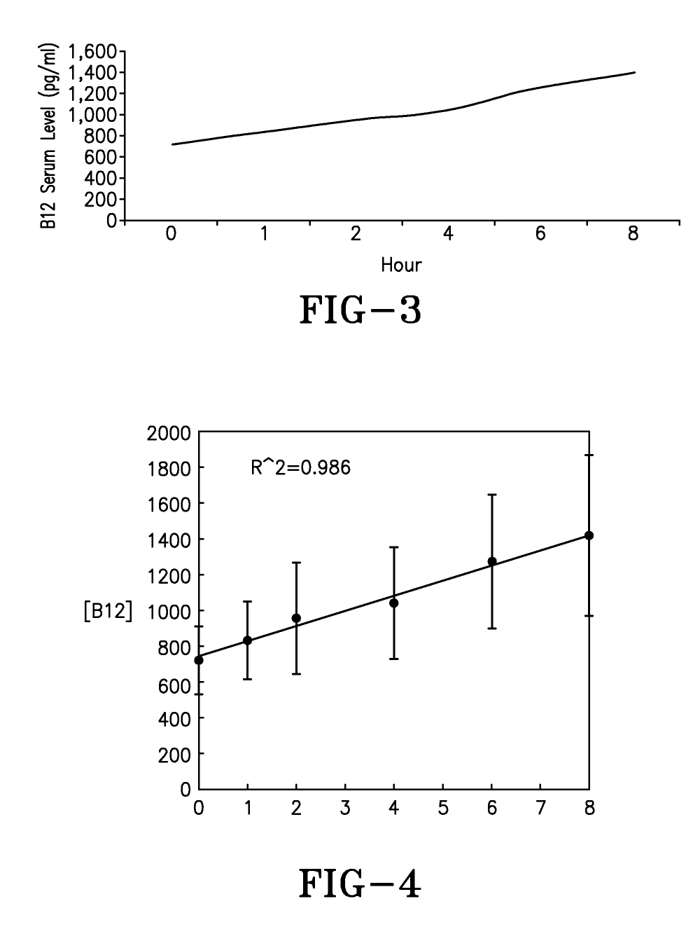

Clinical experiments were performed on four subjects (including two repeats) to obtain a total of six data sets in which vitamin B12 levels in the subject's blood were measured over an 8-hour period. Measurements of vitamin B12 concentration (pg/ml) were taken at the following times (in hours): 0, 1, 2, 4, 6, and 8. The data was averaged between the test subjects and is graphed in FIGS. 3 and 4, with FIG. 4 showing standard deviations.

Analysis of the averaged results shows an approximately linear increase in vitamin B12 blood levels (pg/ml) over the 8-hour period. Analysis of the raw data from this study allows for the following observations.

Vitamin B12 consistently increased in each subject trial over the 8-hour period. At the end of 8 hours, vitamin B12 levels had about doubled, from about 700 pg/ml to about 1,400 pg/ml.

The rate of vitamin B12 delivery varied between subjects by as much as five-fold. The high variation in vitamin B12 absorption suggests such variation can be extrapolated to the general population. With only six test subjects (including four test subjects with 2 repeated trials), the extent and causes of variation in the delivery of vitamin B12 were difficult to ascertain. The causes of variation were likely due to differences in the skin of test subjects; however, variations due to the vitamin B12 patches themselves was ruled out.

A statistical one-way analysis was conducted of the variance of the experimental data, comparing vitamin B12 absorption for the first two hours with the last two hours. The results show the increase in absorption was significant, with a probability of error in rejecting the null hypothesis (that proper absorption was occurring) being about 0.01 (F=7.97). This shows a high statistical significance and indicates that patches 10 enable absorption of vitamin B12 as designed.

Described herein is a transdermal vitamin B12 delivery patch 10, which includes a fabric backing 12, a skin-adhesive polymer matrix 14 on one side of the fabric backing, and a vitamin B12 compound contained within the skin-adhesive polymer matrix, where the vitamin B12 compound diffuses from within the skin-adhesive polymer matrix through the stratum corneum layer of the epidermis into the dermis and into the microvascular of the user. A release liner 16 optionally protects the integrity of skin-adhesive polymer matrix 14 and the vitamin B12 compound therein before patch 10 is adhered to the user's skin.

In particular embodiments of patch 10 of the present invention, permeation enhancers may be employed to improve the diffusion of the vitamin B12 compound through the user's skin. A stabilizer additive may also be employed to retard or prevent UV light-induced degradation of the vitamin B12 compound.

Accordingly, the transdermal vitamin B12 delivery patch of the present invention is simplified, provides an effective, safe, inexpensive, and efficient structure which achieves all the enumerated objectives, provides for eliminating difficulties encountered with prior art vitamin B12 delivery systems, and solves problems and obtains new results in the art.

In the foregoing description, certain terms have been used for brevity, clearness and understanding; but no unnecessary limitations are to be implied therefrom beyond the requirements of the prior art, because such terms are used for descriptive purposes and are intended to be broadly construed.

Moreover, the description and illustration of the invention is by way of example, and the scope of the invention is not limited to the exact details shown or described.

Having now described the features, discoveries and principles of the invention, the manner in which the transdermal vitamin B12 delivery patch is constructed, arranged and used, the characteristics of the construction and arrangement, and the advantageous, new and useful results obtained; the new and useful steps, structures, devices, elements, arrangements, parts and combinations are set forth in the appended claims.

What is claimed is:

1. A transdermal vitamin B12 delivery patch that is applied to the skin of a user for the delivery of vitamin B12 to the bloodstream of the user, said patch comprising:
a fabric backing; and
a skin-adhesive polymer matrix attached to one side of said fabric backing, said matrix containing a vitamin B12 compound, whereby said compound diffuses from the matrix through the stratum corneum layer of the user's skin, through the dermis layer of the skin, and into the user's bloodstream.

2. The transdermal vitamin B12 delivery patch of claim 1, wherein said fabric backing is selected from the group consisting of polyolefins, polyesters, polyethylenes, vinyls, and combinations thereof.

3. The transdermal vitamin B12 delivery patch of claim 2, wherein said fabric is a cross-linked polyolefin foam.

4. The transdermal vitamin B12 delivery patch of claim 1, wherein said skin-adhesive polymer matrix is selected from the group consisting of polyisobutylene, polyoxyl hydrogenated castor oil, ethylene/vinyl acetate copolymer, methacrylate copolymer containing amminio groups, methacrylate copolymer containing amino groups, methacrylate polymer containing carboxyl groups, and mixtures thereof.

5. The transdermal vitamin B12 delivery patch of claim 1, wherein said skin-adhesive polymer matrix includes an acrylate.

6. The transdermal vitamin B12 delivery patch of claim 5, wherein said skin-adhesive polymer matrix includes between about 30% and 85% by weight of a C₄-C₁₂ alkyl acrylate, between about 5% and 50% by weight of a C₁-C₄ alkyl acrylate hardening monomer, and between about 0.4% and 20% by weight of a functionalizing monomer.

7. The transdermal vitamin B12 delivery patch of claim 5, wherein said skin-adhesive polymer matrix includes 2-ethylhexyl acrylate, methyl acrylate, and acrylamide.

8. The transdermal vitamin B12 delivery patch of claim 1, wherein said skin-adhesive polymer matrix further includes at least one permeation enhancer.

9. The transdermal vitamin B12 delivery patch of claim 8, wherein said at least one permeation enhancer is from about 0.001% to about 3% by weight of said polymer matrix.

10. The transdermal vitamin B12 delivery patch of claim 8, wherein said at least one permeation enhancer is selected from the group consisting of nicotinate compounds, fatty acids, fatty alcohols, terpenes, polyols, and cyclic oligosaccharides, and combinations thereof.

11. The transdermal vitamin B12 delivery patch of claim 10, wherein said nicotinate compound is selected from the group consisting of methyl nicotinate, benzyl nicotinate, 2-butoxyethyl nicotinate, isobutyl nicotinate, 1-carbamoylethyl nicotinate, phenyl nicotinate, and n-butyl nicotinate.

12. The transdermal vitamin B12 delivery patch of claim 10, wherein said fatty acid is selected from the group consisting of oleic acid, undecanoic acid, valeric acid, heptanoic acid, pelargonic acid, capric acid, lauric acid, and eicosapentaenoic acid.

13. The transdermal vitamin B12 delivery patch of claim 10, wherein said fatty alcohol is selected from the group consisting of octanol, nonanol, oleyl alcohol and decyl alcohol.

14. The transdermal vitamin B12 delivery patch of claim 10, wherein said terpene is selected from the group consisting of menthol, thymol, limonene, and terpineol.

15. The transdermal vitamin B12 delivery patch of claim 10, wherein said polyol is selected from the group consisting of propylene glycol, polyethylene glycol, and glycerol.

16. The transdermal vitamin B12 delivery patch of claim 10, wherein said cyclic oligosaccharide is selected from the group consisting of alpha cyclodextrin and (1-4)-linked glucopyranose.

17. The transdermal vitamin B12 delivery patch of claim 8, wherein said at least one permeation enhancer includes methylnicotinate and alphacyclodextrin in a concentration of from about 0.001 to 2.0% by weight of said polymer matrix.

18. The transdermal vitamin B12 delivery patch of claim 1, wherein said skin-adhesive polymer matrix further includes a chemical stabilizer suitable for retarding or preventing ultraviolet light-induced degradation of said B12 compound.

19. The transdermal vitamin B12 delivery patch of claim 18, wherein said chemical stabilizer is selected from the group consisting of elemental selenium, sodium selenite, sodium selenate, sodium selenide, dibenzoyl diselenide, selenocystamine, selenobetaine, selenium-methylselenocysteine, selenomethionine, and combinations thereof.

20. The transdermal vitamin B12 delivery patch of claim 18, wherein said chemical stabilizer is from about 0.005 to about 2.0% by weight of said polymer matrix.

21. The transdermal vitamin B12 delivery patch of claim 18, wherein said chemical stabilizer is sodium selenite in a concentration of from about 0.005 to 0.25% by weight of said polymer matrix.

22. The transdermal vitamin B12 delivery patch of claim 1, wherein said vitamin B12 compound is selected from the group consisting of cyanocobalamin, aquacobalamin, hydroxocobalamin, nitritocobalamin, and combinations thereof.

23. The transdermal vitamin B12 delivery patch of claim 1, wherein said vitamin B12 compound is a cyanocobamide.

24. The transdermal vitamin B12 delivery patch of claim 23, wherein said vitamin B12 compound includes 5,6-dimethyl-benzimidazolyl cyanocobamide.

25. The transdermal vitamin B12 delivery patch of claim 1, wherein said vitamin B12 compound includes methylcobalamin, in a concentration of from about 0.25 to about 1.5% by weight of said polymer matrix.

26. The transdermal vitamin B12 delivery patch of claim 1, wherein said polymer matrix contains from about 500 to about 2,000 micrograms of said vitamin B12 compound.

27. The transdermal vitamin B12 delivery patch of claim 26, wherein said polymer matrix contains from about 1,000 to 1,750 micrograms of said vitamin B12 compound.

28. The transdermal vitamin B12 delivery patch of claim 27, wherein said polymer matrix contains from about 1,400 to 1,600 micrograms of said vitamin B12 compound.

29. The transdermal vitamin B12 delivery patch of claim 1, further comprising a release liner, said release liner being removably attached to said polymer matrix and to said fabric backing.

30. The transdermal vitamin B12 delivery patch of claim 29, further comprising an adhesive disposed between said fabric backing and said release liner.
